(19) United States
(12) Patent Application Publication
(10) Pub. No.: US 2008/0235054 A1
(43) Pub. Date: Sep. 25, 2008

(54) DIABETIC CARE PRODUCTS WEB SHOP WITH ENHANCED USABILITY

(76) Inventor: Tan SIEKMANN, Lichtenfels (DE)

Correspondence Address: OSTROLENK FABER GERB & SOFFEN, 1180 AVENUE OF THE AMERICAS, NEW YORK, NY, 100368403, US

(21) Appl. No.: 11/689,796

(22) Filed: Mar. 22, 2007

(51) Int. Cl. A61B 5/00

(52) U.S. Cl. 705/3; 600/300

(57) ABSTRACT

The present invention relates to the field of medical care technology, and in particular to the repeated purchase procedures as it is required for diabetes mellitus patients. In order to increase the usability and user-friendliness for purchasing procedures performed by diabetics when they purchase a new test strip lot, it is proposed to perform the steps of:
a) determining a patient's current consumption of glucose measurement test strips, wherein said test strips form part of said plurality of products,
b) evaluating if said patient can be assumed to have a need for a new lot of test strips within a predetermined time period—e.g. 4 days when the supply time is 2 days,
c) selecting a new lot of test strips matching the patient's glucometer device,
d) initiating the shipping of said new lot automatically to said patient.

### 1. BACKGROUND OF THE INVENTION

1.1. Field of the Invention

The present invention relates to the field of medical care technology.

In particular, it relates to a method, computer program and system for supplying diabetes care products in a system environment including a server computer managing diabetes patient related affairs,

wherein a patient database storing patient-specific data is coupled for cooperation with said server computer,

wherein a product database storing product-specific data for a plurality of said diabetes care products is coupled for cooperation with the server computer,

and a programmed control logic is coupled to the server computer implementing the business steps required for performing individual sales of diabetes care products to individual patients.

1.2. Description and Disadvantages of Prior Art

Although the present invention can be applied to a variety of different applications, it will be set forth and defined from prior art in its special reference to the repeated purchase procedures as it is required for diabetes mellitus patients.

In the year of 2000, more than 151 million people in the world were diabetic. It is predicted that by 2010, 221 million people and by 2025, a number of 324 million will be diabetic.

There are two major forms of diabetes: type 1 and type 2 diabetes. The hall mark of type 1 diabetes is the destruction of insulin producing β-cells in the pancreas, primarily due to autoimmune responses. Type 1 diabetes is manifested with absolute insulin deficiency. In contrast, type 2 diabetes is characterized by two defects: insulin deficiency and insulin resistance. Type 2 diabetes accounts for 90 to 95% of the incidence of diabetes. The current epidemic outbreak of diabetes reflects the high prevalence of type 2 diabetes.

Type-1 diabetes patients usually must measure regularly (several times a day) their glucose level in the blood, for type-2 diabetics this is at least strongly recommended to do it regularly in larger intervals of time. A precise glucose level measurement is key for the appropriate, individual medical care. Thus glucose measurement is a very important concern for many millions of people.

Amongst others problems of managing regular glucose measurements and consuming an appropriate dose of insulin, or sugar, respectively, a diabetic must always take care to have enough medical equipment which allows him to perform the glucose measurements regularly. Basic elements of a glucose measurement system are a glucometer device, some test strips of which generally a single one is required for each measurement, as they are not re-usable, and a calibration stick calibrating the glucose meter device or some electronic measurement evaluation device coupled to the glucometer, e.g., by a Bluetooth interface which outputs a human-interpretable value readily usable for a diagnosis of the current glucose level state.

With particular focus to the present invention a diabetic is thus disadvantageously regularly occupied with the next purchase of a new test strip lot comprising new, unused test strips matching his personal glucometer device in use. It should be added, that there are mostly vendor-specific, and proprietary measurement systems in the market.

In regard of the fact that there is a large plurality of glucometer systems available in the market, the task for a diabetic client to always dispose over an appropriate number of test strips is not simple, in particular for older people having difficulties to recognize and handle the device ID of their glucometer, or, for persons who have basically no time to go for a careful shopping for these new test strips.

FIG. 1 summarizes the steps required in prior art in the upper portion for purchasing a new lot of test strips personally and manually in a respective pharmacy shop for example, whereas the bottom portion of FIG. 1 reflects the situation when using a prior art online shop via the Internet, for example.

Basically, for both cases the person is obliged to observe and control his test strip consumption, step 110. If the test strips tend to run out the person should be aware that he must purchase some new test strip lot in the near future.

With respect to the physical purchase in a pharmacy shop for example the person is constrained to keep its glucometer device ID (GMID in the drawings) available in order to enable the pharmacist to look for a new test strip lot matching exactly the ID of his personal glucometer, step 120.

So, the diabetic patient visits a pharmacy shop in a step 130 and orders, step 140, a new lot of test strips. Then he may pick it up and pay the new lot, if it is available, step 150. In case it is not yet available the diabetic has to repeat this procedure either later in the same pharmacy shop or he may try another shop.

When performing a purchase via the Internet in a virtual pharmacy shop the diabetic will log in into a respective web shop portal, step 160. Then in a further step 165 he may select the product category, for example “diabetes care products”. Then, in a step 170 the diabetic may select the test strip lot matching the GMID of his personal glucometer device. This means in the most cases to select one item from a list of items. Especially, when the user interface implemented in this shop portal is not equipped with user-friendly designed styles and character sizes, this step will impose significant problems to those people having a reduced ability to recognize small characters and list items on their screen. In a next step 175 the person must click to decide for purchasing the new test strip lot, and in a subsequent step 180 he has to perform the payment procedure. Then, if all things go correctly the product will be delivered to him by a respective shipping enterprise.

A person skilled in the daily life of a diabetic person may appreciate that these regularly repeating obligations to purchase a new lot of test strips is quite bothering. An alternative to purchase a respectively higher size test strip lot is not recommendable because the daily use of the glucometer naturally endangers the risk that the device may have some defect and will not be usable any more. When then the diabetic person must decide to purchase a new glucometer device, he will probably select a technically updated one which is may be even cheaper than that one he had in the past. So, he will often have the problem that he cannot use the large quantity of test strips purchased for the dysfunctional, older device.

So, after all, there are many bothering activities required for a diabetic person when he wants to keep the level of available test strips up-to-date with the glucometer device and aligned with a sufficiently large quantity in order to have some reserve quantity in case a purchase will ever fail from whatever reasons.

1.3. Objectives of the Invention

It is thus an objective of the present invention to increase the usability and friendliness for purchasing procedures performed by diabetics when they purchase a new test strip lot.

### 2. SUMMARY AND ADVANTAGES OF THE INVENTION

This objective of the invention is achieved by the features stated in enclosed independent claims. Further advantageous arrangements and embodiments of the invention are set forth in the respective subclaims, which mostly solve further technical objectives. Reference should now be made to the appended claims.

According to the most basic aspect of the present invention, shop server computer managed eventually by a dieabetes care products distributor enterprise sets up some hardware and software equipment technically as known from prior art which implements inventional program components which in turn perform:

a method for supplying diabetes care products in a system environment including a server computer (22) managing diabetes patient related affairs,

wherein a patient database (24) storing patient-specific data is coupled for cooperation with said server computer,

wherein a product database (26) storing product-specific data for a plurality of said diabetes care products is coupled for cooperation with said server computer,

and a programmed control logic is coupled to said server computer implementing the business steps required for performing individual sales of diabetes care products to individual patients,

which method is characterised by performing the steps of:

a) determining a patient's current consumption of glucose measurement test strips, wherein said test strips form part of said plurality of products,

b) evaluating, if said patient can be assumed to have a need for a new lot of test strips within a predetermined time period—e.g. 4 days when the supply time is 2 days,

c) selecting a new lot of test strips matching the patient's glucometer device,

d) initiating the shipping of said new lot automatically to said patient.

The inventional method can be applied basically in situations, in which the diabetic does not use or even possess an electronic telecommunication device and a respective communication channel to the shop server computer associated with the enterprise which offers the products for purchase. In this case of no communication from diabetic to server the server software just needs to store the personal data of a diabetic client including name, address, supply address, glucometer device ID and history data telling the server software at which time at least the last sold lot of test stripes have been send out to the diabetic client. From the stored lot ID the number of sold test strips can be directly derived by a lookup into a product database provided at the server side. Assuming that the consumption of test strips is quite regular for the averaged diabetic client and having stored and ready for a read access the last supply date for the last sold lot of test strips one can easily setup an algorithm for an inventional sales management control program which evaluates the current need for a given diabetic client for a new lot of test strips by estimating the number of consumed test strips and by comparing the resulting estimation count to the total number of test strips available in the diabetic clients current test strip lot. Is then a certain limit reached and the test strips are estimated to run out, then an automatic purchase offer or even shipping procedure can be initiated without establishing any contact to the diabetic client before.

If there is, however, at least a one way information flow possible from the diabetic client to the server and if the diabetic client uses this communication channel in order to send an electronic message for each consumed test strip, then the test strip consumption can be calculated more precisely before the above described automatic offering or shipping is initiated. Thus, the automatically triggered shipping or purchase offer reflects the real, current need for test strips more exactly than if it was based on above mentioned estimation only. Thus, the risk that the diabetic client is not satisfied with the date of the automatically triggered purchase offer or even shipping procedure, is decreased.

If a bi-directional, i.e. two way information flow can be established between the diabetic client, e.g., a Java Applet on a Smartphone or a mobile phone, PC at the client side and the shop server, then a message dialogue can be used in order to further improve the inventional method:

For example after having counted or estimated the diabetic clients test strips and having determined thus the current need of a new test strip lot, an electronic, personal purchase offer message can be sent out to the client asking him just to acknowledge this message in order to trigger the purchase and shipping of a new test strip lot. On receipt of such acknowledgement message from the client the purchase procedure and shipping can be performed automatically without any further human intervention. Thus, the inventional method generally helps to reduce the cost at the vendor side significantly.

The before mentioned embodiment of the inventional method can be further enriched by including a message which includes the calibration data for the purchased new test strip lot in an electronically readable form. By that the advantage results that the diabetic person has all relevant information to calibrate the measurements performed with the new test strips, right in a form, which allows immediate readily usable calibration of glucose measurement data in particular in cases as described in the Applicant's copending patent application EP 06006190, which is incorporated herein by reference.

The before mentioned embodiment of the inventional method can be further enriched by including a message dialogue which is directed to a just the supply date for the next new lot to the current need of the diabetic client. This can be helpful in situations in which the client decides to go on holidays for a couple of weeks or is at least absent from his usual supply address. Further, it can be helpful to readjust a time delay which may slowly come in over the time representing a deviation between the server side estimation or count of consumed test strips and the reality at the client. In order to do that it is proposed to send an electronic “just-in-time-supply” control message to the patient's electronic communication device which offers him an easy-to-use input interface for defining a time delay or a time advance which the client may wish to implement in this automatic supply procedure. The server then receives the respective response message may evaluate the number of days for moving the shipping date for the next new test strip lot and may complete the procedure accordingly.

The client telecommunication device may be for example a mobile phone, or a smart phone which implements for example a java applet which performs the above described client-side method steps. For the communication per se the usual prior art available protocols can be used, for example SMS, MMS, if required or even an oral communication can be performed accompanied with a speech recognition system at the server-side or the server-side starve member. The client side electronic communication device may also be a personal computer connectable to the internet. In this case the server-side server computer comprises a web server software as known from prior art and implementing the technical communication interface and the correct business logic for performing above mentioned method steps.

### 3. BRIEF DESCRIPTION OF THE DRAWINGS

The present invention is illustrated by way of example and is not limited by the shape of the figures of the drawings in which:

FIG. 1 illustrates the control flow of the most important steps of two variants of a prior art method performed when purchasing a new test strip lot;

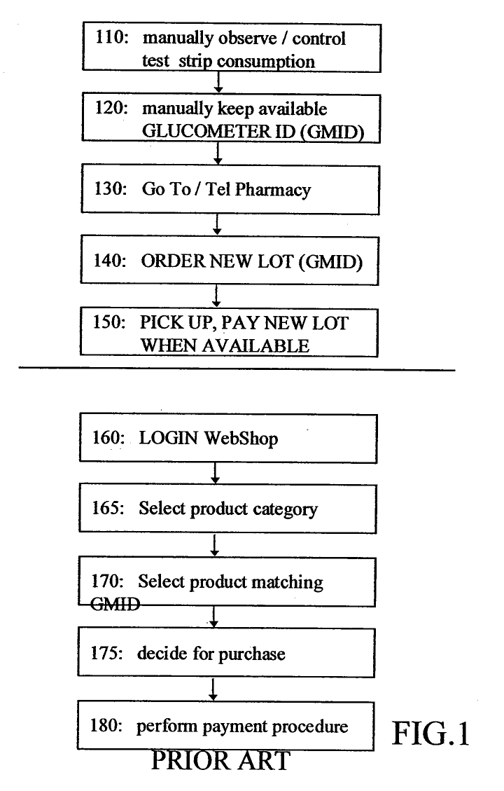

FIG. 2 illustrates the most basic structural components of a inventional hardware and software environment used for a preferred embodiment of the inventional method,

FIG. 3 illustrates the control flow of the most important steps of a preferred embodiment of the inventional method, performed at the shop server side,

FIG. 4 illustrates the control flow of the most important steps of a further preferred embodiment of the inventional method, performed at the shop server side, modified by an additional “just-in-time-supply” message dialogue; and

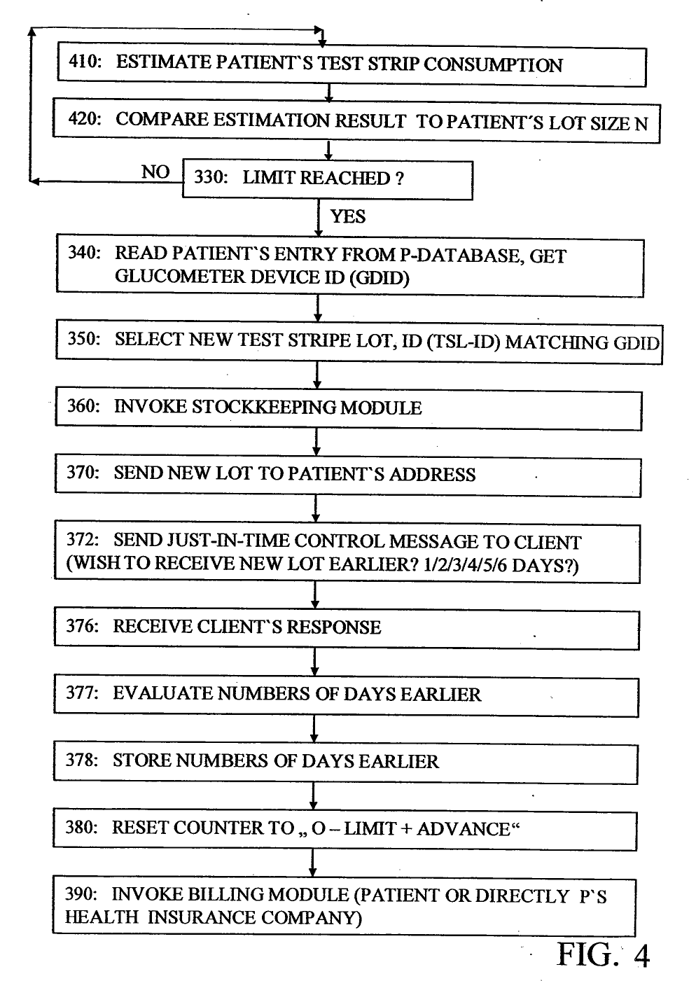

FIG. 5 illustrates a dataset stored for a specific client in the server-side client's database.

### 4. DETAILED DESCRIPTION OF THE PREFERRED EMBODIMENT

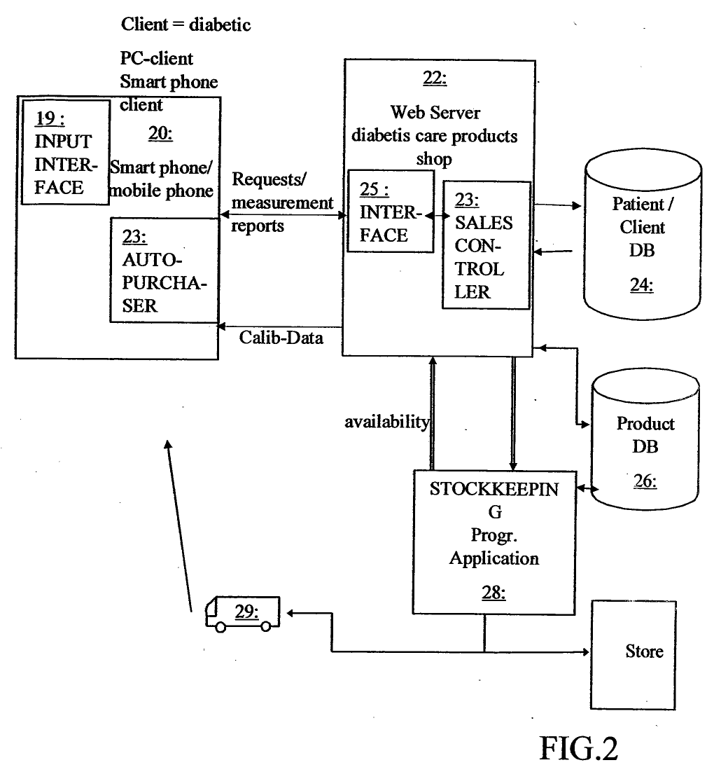

With general reference to the figures and with special reference now to FIG. 2 the client side is depicted at the left margin of the drawing including a mobile phone 20 according to prior art having implemented a java applet 23 which implements the basic steps required for the client in order to perform a message dialogue with the server and which implements an input interface 19 for recording the number of consumed test strips per lot. A preferred implementation of the input interface 19 comprises a Bluetooth interface which communicates to a respective Bluetooth interface installed at a glucometer device itself. Such interface is described in more detail in a co pending European patent application EP06006190. This patent application is incorporated herein be reference. Via this Bluetboth interface preferably every time when the diabetic performs a glucose measurement and thus consumes one test strip the glucometer device user for the measurement sends a dataset to a smart phone or a mobile phone such as mobile phone 20 of FIG. 2. A sample implementation of such dataset is described in the above referenced European patent application. Such dataset can be advantageously used also for the purpose of the present invention because it comprises amongst other data fields the current date and time of the current glucose measurement. Further, advantageously due to the fact, that each time a measurement is done the mobile phone 20 receives some dataset via its input interface 19, the automatic purchaser applet 23 implemented at the mobile phone is enabled to forward this “measurement-event” to the web server 22 at the server side (depicted at the right portion in FIG. 2), which may serve as a basic input for counting the consumed test strips and thus to determine a suited point in time when to trigger the next automatic purchase procedure. The smart phone of the diabetic client has prior art send and receive means in order to initiate and perform the sending and receiving, respectively, of messages in cooperation and communication with the web server 22 and in particular with its respective communication interface parts 25.

With further reference to FIG. 2 the web server 22 and the software components installed there implement a diabetes care products shop which can be communicated with via internet based communication, mobile communication and preferably cable bound telephone technology. Respective technical input and output interface means 25 implement a respective communication protocol. Further preferably, an interface is provided in order to be served by starve members when they communicate via public telephone network. So, for each communication channel, a respective communication server is implemented in the general interface 25 of the server computer. It should be noted, that this functionality can be also distributed over a plurality of physical hardware, i.e. a plurality of respective server computer devices.

With respect to the implementation of the inventional steps implementing the more business related aspects of the inventional method, a sales management control program component 21 is provided which has input and output interfaces to the general interface 25, as well as input and output interfaces to a client database 24 as well as input and output interfaces to a commissioning program application 28 which manages the physical store of the products being offered, as well as input and output interfaces to a product database 26.

With further respect to the client database 24 it is recommended to implement this database as a relational database and use any prior art database management system in order to read and write data from or to the database, respectively.

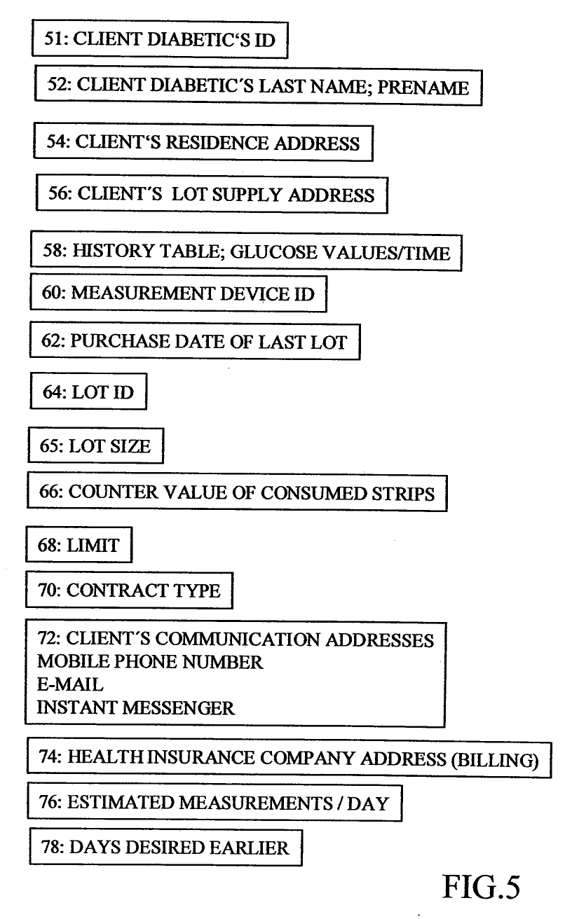

With reference to FIG. 5 an exemplary dataset is depicted which comprises the data fields stored for each client as follows: A field 52 comprising name, field 54 comprising the address for billing purposes, field 56 comprising the address implementing the shipping destination for a purchased product a field 58 comprising a pointer to a history table which stores further, medical data possibly relevant for further medical consultancy services and possibly including historic values of measured glucose values accompanied with respective date and time indications, a glucometer device ID 60 which tells the identity of the glucometer in use and which is used as a basic information for a later selection of the proper and matching test strip lot, a field 62 comprising the date of the last purchased new test strip lot, a field 64 comprising an identification key for the lot which was last purchased by the client at the date of field 62, a field 65 comprising the number of test strips comprised of the last purchased lot in field 62, a further field 66 comprising a counter value representing the number of consumed test strips preferably related to the last purchased lot of field 64, a data field 68 comprising a personal limit, which is a count representing the limit that a certain, predetermined minimum number of test strips is available at the diabetic client, and a data field 70 comprising a flag describing the type of contract between diabetic client and test strip distributor enterprise using the inventional method.

With reference back to FIG. 2 the product database 26 can be read from or written to by the sales management control program 21. The product database 26 is preferably implemented also as a relational database which may be also accessed via a respective database management system. With respect to the very focus of the present invention the product database 26 has a plurality of tables which store for different glucometer devices the matching test strip lot ID. By this feature, a quick reference can be done for selecting the correct test strip lot matching the diabetic client's glucometer device. Of course, several lot sizes are stored and respective calibration data for each test strip lot as well as a respective calibration data ID are stored in respective tables. For the best implementation how to store those data, prior art database management gives a sufficient teaching for optimized storage and handling of the above described data.

The stock keeping program application 28 is used for implementing the interface to the physical store management in order to keep the available number of products for each product ID and product category updated with the reality prevailing at the physical store. So, a prior art warning can be generated by the stock keeper program 28 when some product is not available for the moment. Thus, the controller program 21 may include some special logic in order to select for each non-available product a second product which may be used instead of the not available one and has similar properties.

A shipping service symbolized by lorry 29 is used for shipping the purchased product to the diabetic client's supply address.

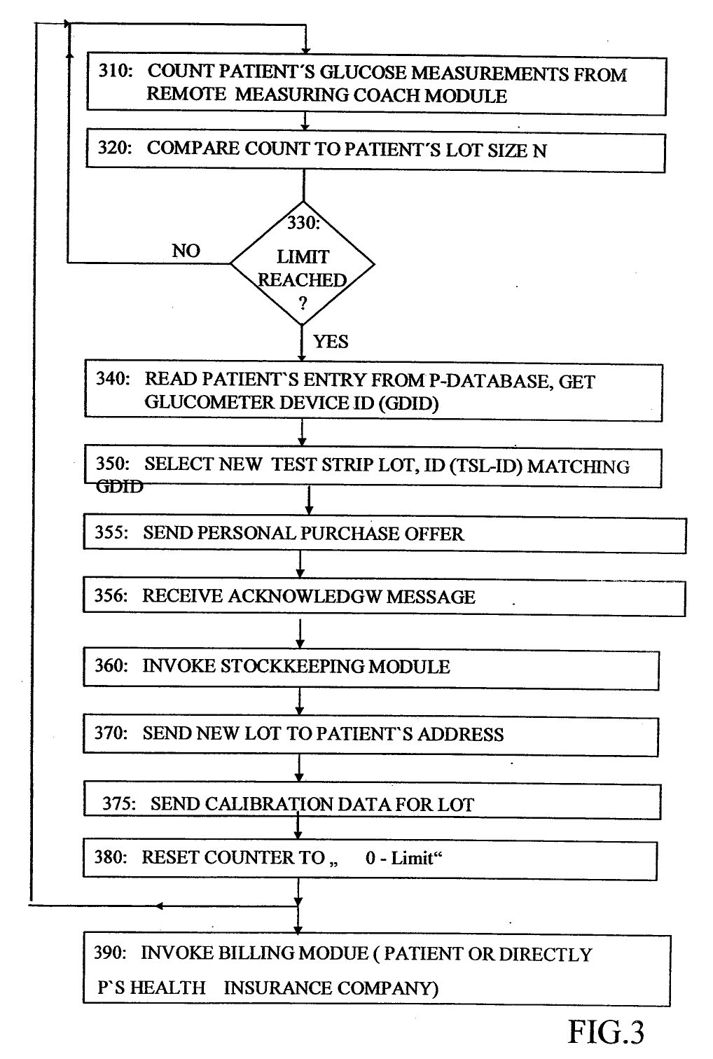

With further reference to FIG. 3 the control flow within the server computer 22 will be described in more detail when a preferred embodiment of the inventional method is performed.

In this sequence of steps a bi-directional communication is performed between the communication device of the diabetic client and the server computer.

In a first step a count loop is entered, referred to with reference sign 310 in which the patient's measurements of the glucose values and so the number of consumed test strips is counted. In order to be able to count those events the client's mobile phone implements within the applet 23 a further program component referred to in here as “client measuring coach module”, which sends each time when a measurement was performed and the client's mobile phone received a measured value via its Bluetooth interface to the glucometer device, as disclosed in the above mentioned co-pending patent application, a message automatically to the communication interface 25 of the server computer 22. Thus, each of such incoming message reflecting the fact that a glucose test strip has been consumed, increases the current value of a counter by “1”. Then, in a further step 320 the current counter value is compared with the number of unused test strips remaining at the diabetic client. This value can be retrieved easily by once looking up in the client database 24 the field 65, which stores this original number of test strips as “lot size”, by lot size minus current counter value.

Then, the pre-defined data field 68 storing the notification limit is looked up from the patient database and it is calculated if the limit is reached or is not yet reached. Say, for example the lot size is N=100 (original amount of test strips in a new, unused lot),

the counter value is currently increased to “85” and the predefined limit has a value of “12”, then the calculation reads as follows:

100−85=15

If this calculated value, which corresponds to the number of unused test strips available for the client for future measurements, is smaller than the limit of 12, then the Yes-branch of decision 330 will be entered as the limit has been reached. To reach the limit means that some action will be undertaken automatically in order to trigger an automatic purchase procedure without major intervention being required for the diabetic client.

In the No-branch of decision 330, the control is fed back to step 310 in order to reenter the loop and wait for the next increase of the counter.

Then, in a further step 340, following the Yes-branch of decision 330 and in order to initiate the automatic purchase procedure the sales management control program 21 located at the server computer reads the current patient's entry from the patient database in order to look up the glucometer device ID (GDID) currently in use. This entry will be identified by the diabetic client's ID which is always part of each incoming message from a client's electronic communication device.

Then, in a next step 350 the sales management control program 21 selects the ID-tag for the next new test strip lot (TSL-ID) which matches the patient's glucometer device ID. This is basically done by a simple look up into the product database 26, wherein the glucometer device ID is used as an index for example.

In a simple variant of this embodiment the next steps 355 and 356 are omitted.

In a more customizable version of this preferred embodiment in the step 355 the server computer sends a purchase offer to the client's telecommunication device. This purchase offer message is personal in nature because it is sent specifically to the current client and reflects his specific needs.

The client's communication address is also stored in the dataset depicted in FIG. 5, see data fields 72. Basically, any of those stored communication addresses may be used. Preferably a priority indicating sequence of communication channels is also stored in this data field. So for example, first, the patient's mobile phone is used as communication target and a SMS message is sent to him offering him to purchase a new test strip lot just by pressing some key on its mobile phone. Then, the key pressing will trigger automatically the return message to the server computer using the same communication channel having the business meaning of an acknowledgement, i.e. acceptance of the purchase offer.

This response message is then received in a step 356 at the server computer.

Further preferably, if a response message is not received within a predetermined time limit the server undertakes a second trial using a different communication channel, for example to send a predefined e-mail or an instant messenger message to the client. Again, a respective address stored in the data fields 72 is used for this purpose.

This procedure is continued until some response has been received from the client. The time intervals used between different repetitions can be predefined. A good value is around one or two hours and leaving out the night times where a person can be assumed to sleep. Then, after having received an acknowledgement message for the automatic purchase, the stock keeping module 28 is invoked at the server side in a step 360 which in turn initiates the shipment of the selected new lot to the patient's address stored in data field 56, FIG. 5.

Then, preferably a further step 375 is performed, in which the server computer looks up the calibration data stored also in the product database for the selected new lot of test strips and sends this data to the mobile phone of the client which is processed by a respective applet installed there in order to calibrate the measured values when they come in freshly from the glucometer device.

It should be added, that the new calibration data has some identification tag which must match an electronic marker implemented on each test strip. So, it is basically impossible to use the wrong calibration data for a given test strip. Further, preferably, the user is prompted by its Java applet running on its mobile phone when the mobile phone detects a new test strip lot ID for the first time. Then, the user is aware that the Java applet controlling the calibration of measuring data now assumes that a new test strip lot has been opened and the first test strip thereof has been in use.

The procedure to send calibration data for some test strip lot is also described in the above mentioned European patent application of the same applicant.

Then, in a further step 380 the counter is reset to a value of “0-Limit”, in order to re-begin counting in an appropriate way for reflecting that the new consumption of test strips counts relative to the new test strip lot corresponds to a value of “0”, when the client diabetic has consumed the last test strip of his old test strip lot.

Then, control is fed back to step 310 in order to wait for the new measurement event. Concurrently to that, a billing module as it is known from prior art will be invoked in a step 390. Preferably and adjusted to this specific use case, the billing module will send the bill either to the patient or directly to the patient's health insurance company. Those alternatives are predefined in the personal dataset of the patient, see data field 74.

A further preferred embodiment of the inventional method includes a section of a workflow at the server computer 22 which helps to perform the automatic purchase procedure without exact, counted information from the client diabetic's telecommunication device being present, telling the server about the number of used test strips.

In this embodiment, in a step 410, a loop is entered which estimates the patient's test strip consumption. In order to do that, the sales management control program 21 according to this embodiment looks up the patient/client database 24 for reading data field 62 telling about the date of the lastly purchased new lot, further the data field 65, telling about the lot size of the lastly purchased new lot, and a data field 76 which stores a number indicating the number of measurements per day which the diabetic client is assumed to perform. So, in this field usually a value between 1 and 3 will be stored. Then, the control program 21 reads the current date and will calculate:

M=L−(today−last purchase date)[days]*f

Wherein:

L is the lot size 65,

today—last purchase date is the number of days since the lastly purchased new lot, see data field 62,

f is a frequency and is read from data field 76 representing the number of measurements per day assumed for the client,

M is the number of consumed test strips of the current lot.

Then, basically this estimation value is used for the decision 330, as in FIG. 3 if or if not to trigger a new automatic purchase procedure. Then, basically the same control flow is followed as described above for steps 340, 350, 360, 370. Then, after in step 370 the new lot has been sent out to the patient's shipping address, in a further sequence of steps specifically useful in this situation a procedure is performed which helps to assure that the estimation performed in step 410 and 420 is not too bad compared to the reality prevailing at the client diabetic.

In order to do so, in a step 372 a preferred implementation sends out a so-called just-in-time-control message to the client via any of the available communication channels. Basically, if no electronic communication channel exists, even a short letter can be shipped to the client, including a prepared response letter form. The content of such control message is to ask from the client if he wishes to receive the next new lot of test strips earlier and if yes, to let the client diabetic specify the number of days the shipping of a new lot is desired earlier. In case this is an electronic just-in-time-control message, for example a SMS sent out to a mobile phone of the client, the cursor in the display message can be pre-positioned into a data field which selects one of a given list of data fields, for example one day earlier, two days earlier, three days earlier, and so on in order to give the diabetic client an input means very simply to use, which enables him to directly select the number of days he desires to receive the next new test strip lot earlier than planned in the schedule based on the current estimation.

Then, the client can be assumed to press the correct day indication field and will thus specify this information. Alternatively, one can offer an input field to the client asking him to input the number of test strips available at his site for this specific glucometer device. Then the respective response divided by the number of measurements delivers a base to estimate the number of days the diabetic client may stay without having a new test strip lot.

Again, the pressing of the key can be used to trigger a response message using the same communication channel as used for the sending of the just-in-time-control message in step 372.

Then, in a further step 376 the client's response message is received at the server. Further, in step 377 the “number of days earlier” is read from the message and is stored in step 378 into data field 78. This information is then used in the next estimation step 410 for producing a more exact estimation of the consumed test strips. Thus, in a preferred embodiment the limit calculation step 330 above is updated with the value of the data field 78. After usage of data field 78, this field is tagged to be already used. Before it is re-used in future, it must be freshly re-written by means of a “fresh” message from the client, (step 376). The respective plurality of missing test strips corresponding to the “days-earlier” indication can be easily calculated by multiplying data field 78 with the value of data field 76.

The invention can take the form of an entirely hardware embodiment, an entirely software embodiment or an embodiment containing both hardware and software elements. In a preferred embodiment, the invention is implemented in software, which includes but is not limited to firmware, resident software, microcode, etc.

Furthermore, the invention can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

1. A method for supplying diabetes care products in a system environment including a server computer managing diabetes patient related affairs,
wherein a patient database storing patient-specific data is coupled for cooperation with said server computer, wherein a product database storing product-specific data for a plurality of said diabetes care products is coupled for cooperation with said server computer, and a programmed control logic is coupled to said server computer implementing the business steps required for performing individual sales of diabetes care products to individual patients,
wherein by the steps of:
a) automatically determining a patient's current consumption of glucose measurement test strips, wherein said test strips form part of said plurality of products,
b) automatically evaluating, if said patient can be assumed to have a need for a new lot of test strips within a predetermined time period,
c) automatically selecting a new lot of test strips matching the patient's glucometer device,
d) automatically initiating the shipping of said new lot automatically to said patient.

2. The method according to claim 1, wherein information from which the current number of currently consumed test strips of a predetermined lot of test strips can be derived automatically without human intervention, further comprising the step of:
evaluating said need for a new lot of test strips by
estimating the number of consumed test strips by a predetermined rule and comparing a resulting estimation count to the total number of test strips available in the patient's current lot.

3. The method according to claim 1, wherein an interface is provided for receiving electronic messages sent by an electronic telecommunication device associated with a patient, wherein a message comprises information from which the current number of currently consumed test strips of a predetermined lot of test strips can be derived automatically without human intervention, further comprising the step of:
evaluating said need for a new lot of test strips by comparing the number of consumed test strips and comparing the count to the total number of test strips available in the patient's current lot.

4. The method according to claim 3, wherein an interface is provided for receiving and sending electronic messages from and to, respectively, an electronic telecommunication device associated with a patient, further comprising the step of:
a) sending an electronic “just-in-time-supply” control message to the patient's electronic communication devices,
b) receiving a response message, evaluating said response message for adjusting a future supply date relevant for a respective future supply of a new test strip lot.

5. The method according to claim 4, wherein said electronic telecommunication device associated with a patient is selected from the group of:
a) a mobile phone,
b) a Smartphone,
c) a Personal computer.

6. The method according to claim 4, further comprising the step of:
sending a message to the client including the calibration data in an electronically readable form for the purchased new test strip lot.

7. The method according to claim 1, further comprising the step of:
a) sending a “just-in-time-supply” control message to said electronic communication device,
b) receiving a response to said “just-in-time-supply” control message,
c) evaluating said response, and
d) adjusting a time schedule to be evaluated for a next supply shipment of said test strips.

8. A method for electronically purchasing diabetes care products in cooperation with a system environment including a server computer managing diabetes patient related affairs,
wherein a patient database storing patient-specific data is coupled for cooperation with said server computer, wherein a product database storing product-specific data for a plurality of said diabetes care products is coupled for cooperation with said server computer, and a programmed control logic is coupled to said server computer implementing the business steps required for performing individual sales of diabetes care products to individual patients,
wherein by the steps of:
a) receiving a “just-in-time-supply” control message from said server computer,
b) displaying an input control for receiving a user input for generating an electronic response to said received “just-in-time-supply” control message,
c) sending said response in a response message to said server computer.

9. An electronic data processing system implemented for supplying diabetes care products in a system environment including:
a server computer managing diabetes patient related affairs,
a patient database storing patient-specific data and coupled for cooperation with said server computer,
a product database storing product-specific data for a plurality of said diabetes care products and coupled for cooperation with said server computer,
and a programmed control logic coupled to said server computer implementing the business steps required for performing individual sales of diabetes care products to individual patients,
wherein by:
a) means for automatically determining a patient's current consumption of glucose measurement test strips, wherein said test strips form part of said plurality of products,
b) means for automatically evaluating, if said patient can be assumed to have a need for a new lot of test strips within a predetermined time period,
c) means for automatically selecting a new lot of test strips matching the patient's glucometer device,
d) means for automatically initiating the shipping of said new lot automatically to said patient.

10. A computer program product comprising a computer useable medium including a computer readable program, wherein the computer readable program includes a functional component that when executed on a computer causes the computer to perform the steps of:
a) automatically determining a patient's current consumption of glucose measurement test strips, wherein said test strips form part of said plurality of products,
b) automatically evaluating, if said patient can be assumed to have a need for a new lot of test strips within a predetermined time period,
c) automatically selecting a new lot of test strips matching the patient's glucometer device,
d) automatically initiating the shipping of said new lot automatically to said patient.

11. The method according to claim 4, further comprising the step of:
a) sending a “just-in-time-supply” control message to said electronic communication device,
b) receiving a response to said “just-in-time-supply” control message,
c) evaluating said response, and
d) adjusting a time schedule to be evaluated for a next supply shipment of said test strips.
